(19) United States
(12) Patent Application Publication
(10) Pub. No.: US 2022/0228216 A1
(43) Pub. Date: Jul. 21, 2022

(54) Methods, Compositions, and Systems for Detecting Silent Carriers of Spinal Muscular Atrophy

(71) Applicant: Laboratory Corporation of America Holdings, Burlington, NC (US)

(72) Inventors: Patricia Okamoto, Shrewsbury, MA (US); Zhenxi Zhang, Westborough, MA (US)

(21) Appl. No.: 17/576,188

(22) Filed: Jan. 14, 2022

Related U.S. Application Data
(60) Provisional application No. 63/137,889, filed on Jan. 15, 2021.

(51) Int. Cl. C12Q 1/6883; C12Q 1/6858; C12Q 1/686

(52) U.S. Cl. CPC C12Q 1/6883; C12Q 1/6858; C12Q 2600/16; C12Q 2600/156; C12Q 1/686

(57) ABSTRACT

The present disclosure relates to methods, compositions, and systems for detecting silent carriers of spinal muscular atrophy (SMA). In some embodiments, the invention comprises a method for identifying a subject as a silent carrier of SMA. The method may comprise obtaining a nucleic acid sample from a subject. The method may further comprise analyzing the nucleic acid sample, wherein analyzing the nucleic acid sample comprises detecting the presence or absence of a target gene amplification product. The method may further comprise characterizing the subject as a silent carrier of SMA if the target gene amplification product is present. Also disclosed are systems and kits for carrying out embodiments of the methods or using the compositions disclosed herein.

### RELATED APPLICATIONS

This application claims priority to U.S. Provisional Patent Application No. 63/137,889, filed Jan. 15, 2021. U.S. Provisional Patent Application No. 63/137,889 is incorporated by reference in its entirety herein.

### FIELD OF INVENTION

This application is directed to methods, compositions, and systems for detecting silent carriers of spinal muscular atrophy (SMA).

### BACKGROUND

Spinal muscular atrophy (SMA) is a common autosomal-recessive disease mainly caused by a homozygous deletion of the survival motor neuron 1 (SMN1) gene on chromosome 5q13.2. Current SMA carrier screening methods look for SMN1 copy number losses. In some cases, however, there is duplication of the SMN1 gene, such that certain individuals may have two copies (or more) of the SMN1 gene on one chromosome 5 and no SMN1 copies on the other chromosome 5 (denoted 2+0). Current methods are unable to determine the number of SMN1 copies present on individual chromosomes and as such, do not distinguish an individual with two SMN1 copies on one chromosome and no copies on the other (2+0, silent carrier) from an individual who has one SMN1 gene on both chromosomes (1+1). Thus, silent carriers are not identified as a carrier by traditional screening methods and receive a false-negative test result. Testing methods with the capability to identify these SMA silent carriers are expensive, time-consuming, and of questionable accuracy. Thus, there is a need in the art for an efficient, accurate assay to detect silent carriers of SMA.

### SUMMARY

The present disclosure relates to methods and systems for detecting silent carriers of spinal muscular atrophy (SMA).

In some embodiments, the invention comprises a method for identifying a subject as a silent carrier of SMA. The method may comprise obtaining a nucleic acid sample from a subject. The method may further comprise analyzing the nucleic acid sample, wherein analyzing the nucleic acid sample comprises detecting the presence or absence of a target gene amplification product. The method may further comprise characterizing the subject as a silent carrier of SMA if the target gene amplification product is present. In some embodiments, the method of analyzing the nucleic acid sample may comprise a real-time PCR assay. In some embodiments, the method of analyzing the nucleic acid sample may comprise analyzing exon 7 of SMN1 or a portion thereof. In some embodiments, the method of analyzing the nucleic acid sample may comprise analyzing rs143838139 in the SMN1 gene amplification product to detect the presence of a T>G mutation. In some embodiments of the method, a mutant SMN1 gene amplification product is detected with a labeled nucleic acid probe specific for the mutant SMN1 gene amplification product. In some embodiments, of the method, the probe is labeled with a fluorophore. In some embodiments of the method, Ct is determined the fluorescent dye channel after the data is collected, and the presence or absence of the allele is determined by whether the curve Ct is lower than a predetermined threshold.

In other embodiments, the invention comprises a system for identifying a subject as a silent carrier of SMA. In some embodiments, the system comprises components for carrying out the methods of the invention disclosed herein.

In yet other embodiments, the invention comprises compositions or kits for identifying a subject as a silent carrier of SMA. In some embodiments, the kit comprises compositions and/or other components for carrying out the methods of the invention disclosed herein.

### BRIEF DESCRIPTION OF THE DRAWINGS

FIG. 1 depicts a schematic of the expression of SMN protein a healthy individual and a schematic of the lack of expression of SMN protein in an SMA patient.

FIG. 2 depicts examples of the different disease and carrier statuses associated with the copy number of SMN1 on each allele.

FIG. 3 depicts a method for detecting an SMA silent carrier in accordance with an embodiment of the disclosure.

FIG. 4 depicts a system for detecting an SMA silent carrier in accordance with an embodiment of the disclosure.

### DETAILED DESCRIPTION

The following description recites various aspects and embodiments of the present invention's methods, compositions, and systems. No particular embodiment of the invention is intended to define the scope of the methods and systems. Rather, the embodiments merely provide non-limiting examples of various methods and systems that are at least included within the scope of the invention. The description is to be read from the perspective of one of ordinary skill in the art; therefore, information well known to the skilled artisan is not necessarily included.

### Definitions

The present invention now will be described more fully hereinafter. The invention may be embodied in many different forms and should not be construed as limited to the aspects set forth herein; rather, these aspects are provided so that this disclosure will satisfy applicable legal requirements. Unless defined otherwise, all technical and scientific terms used herein have the same meaning as is commonly understood by one of ordinary skill in the art to which this invention belongs. All patents, applications, published applications and other publications referred to herein are incorporated by reference in their entireties. If a definition set forth in this section is contrary to or otherwise inconsistent with a definition set forth in the patents, applications, published applications and other publications that are herein incorporated by reference, the definition set forth in this section prevails over the definition that is incorporated herein by reference.

When introducing elements of the present invention or the embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. It is understood that aspects and embodiments of the invention described herein include “consisting” and/or “consisting essentially of” aspects and embodiments.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

Various aspects of this disclosure are presented in a range format. It should be understood that the description in range format is merely for convenience and brevity and should not be construed as an inflexible limitation on the scope of the disclosure. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

“Sample” or “tissue sample” or “patient sample” or “patient cell or tissue sample” or “specimen” each refer to a collection of similar cells obtained from a tissue of a subject or patient. The source of the tissue sample may be solid tissue as from a fresh tissue, frozen and/or preserved organ or tissue or biopsy or aspirate; blood or any blood constituents (e.g., plasma or serum), cell free DNA, RNA, bodily fluids such as cerebral spinal fluid, amniotic fluid, peritoneal fluid or interstitial fluid or cells from any time in gestation or development of the subject. The tissue sample may contain compounds that are not naturally intermixed with the tissue in nature such as preservatives, anticoagulants, buffers, fixatives, nutrients, antibiotics or the like. Cells may be fixed in a conventional manner, such as in an FFPE manner. Also, samples may be dried for subsequent transfer and/or analysis (e.g., dried blood or dried plasma).

As used herein, the terms “individual,” “subject” and “patient” are used interchangeably. As used herein, the terms “subject” and “subjects” refer to an animal, preferably a mammal including a non-primate (e.g., a cow, pig, horse, donkey, goat, camel, cat, dog, guinea pig, rat, mouse or sheep) and a primate (e.g., a monkey, such as a cynomolgus monkey, gorilla, chimpanzee or a human).

As used herein, the term “detectable moiety” or “detectable biomolecule” or “reporter” refers to a molecule that can be measured in a quantitative assay. For example, a detectable moiety may comprise an enzyme that may be used to convert a substrate to a product that can be measured (e.g., a visible product). Or, a detectable moiety may be a radioisotope that can be quantified. Or, a detectable moiety may be a fluorophore. Or, a detectable moiety may be a luminescent molecule. Or, other detectable molecules may be used.

The terms “labeled” and “labeled with a detectable agent or moiety” are used herein interchangeably to specify that an entity (e.g., a nucleic acid probe, antibody, etc.) can be measured by detection of the label (e.g., visualized, detection of radioactivity and the like) for example following binding to another entity (e.g., a nucleic acid, polypeptide, etc.). The detectable agent or moiety may be selected such that it generates a signal which can be measured and whose intensity is related to (e.g., proportional to) the amount of bound entity. A wide variety of systems for labeling and/or detecting nucleic acids are known in the art. Labeled nucleic acids can be prepared by incorporation of, or conjugation to, a label that is detectable by spectroscopic, photochemical, biochemical, immunochemical, electrical, optical, chemical or other means. A label or labeling moiety may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionucleotides, fluorophores, chemiluminescent agents, microparticles, enzymes, colorimetric labels, magnetic labels, haptens, molecular beacons, aptamer beacons, and the like.

### Methods

In some embodiments, the invention comprises methods for identifying a subject as a silent carrier of spinal muscular atrophy (SMA). The methods may be embodied in a variety of ways.

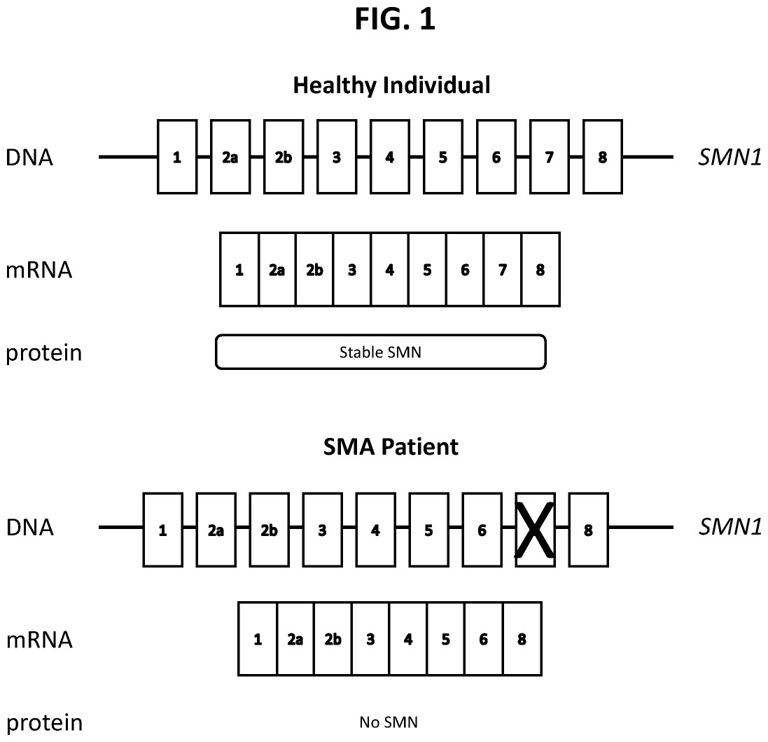

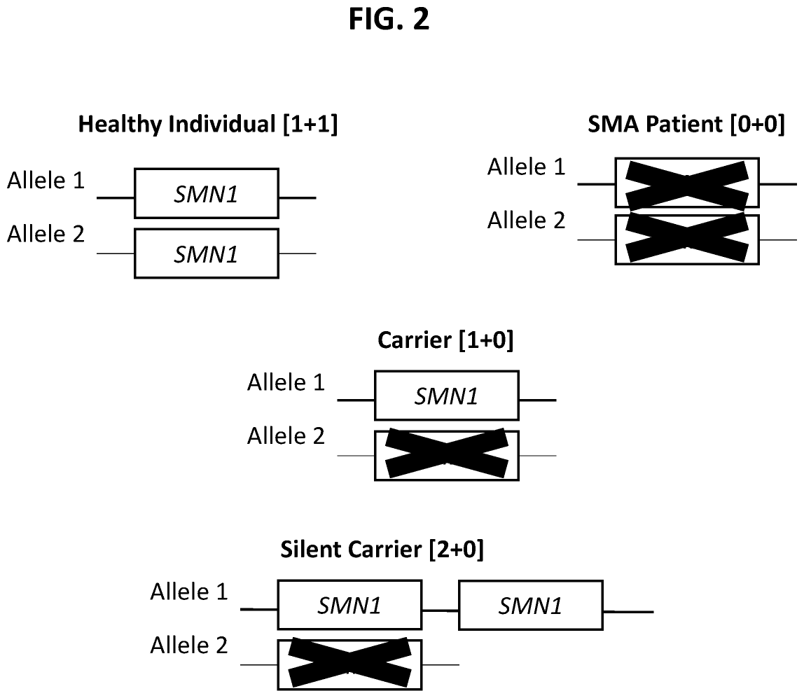

SMA is an autosomal-recessive disease with a carrier frequency ranging from about 1 in 35 to 1 in 117, depending on ethnicity. The disease results in bilateral muscle weakness and eventually atrophy caused by the progressive degeneration and loss of anterior horn cells in the spinal cord. The most common cause of SMA is a homozygous deletion of the survival motor neuron 1 (SMN1) gene on chromosome 5q13.2 as is depicted in FIG. 1; in up to 98% of SMA patients, both copies of SMN1 are either deleted or rendered non-functional. As most mutations causing SMA involve SMN1 copy-number loss, carrier screening commonly involves determining the number of SMN1 copies an individual has. As is shown in FIG. 2, people with at least one SMN1 copy on each chromosome (1+1) are not SMA carriers. Current screening methods can readily identify an SMA carrier with an SMN1 copy on one chromosome and no SMN1 copy on the other (1+0). However, current screening methods will provide a false negative result for an individual with two (or more) copies of SMN1 on one chromosome and no copies of SMN1 on the other chromosome (2+0). In the present disclosure, the term “silent carrier” refers to an individual with two or more copies of SMN1 on one chromosome and no copies of SMN1 on the other chromosome. These silent carriers have a 50% chance of passing the null allele to their progeny.

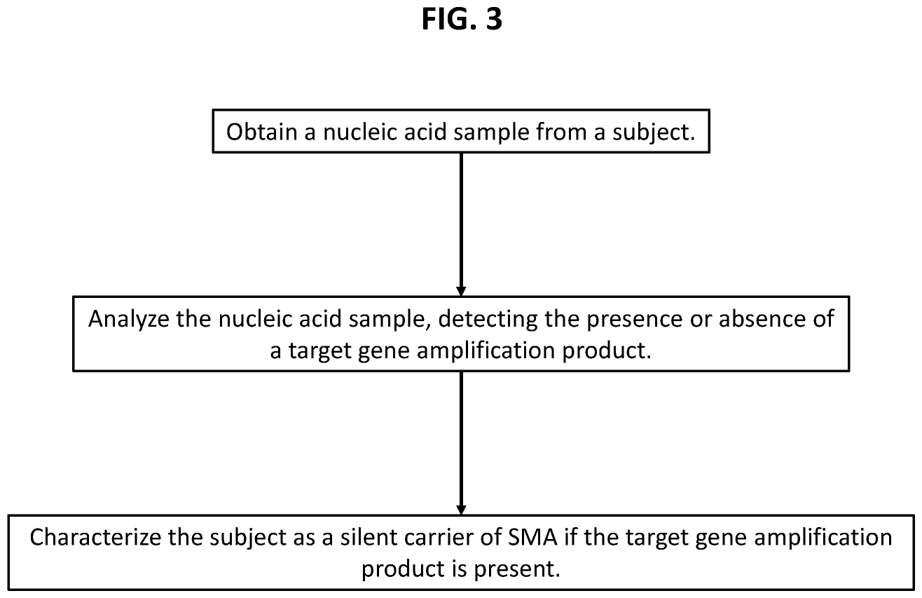

Therefore, in some embodiments, the invention comprises a method for identifying a subject as a silent carrier of SMA. An embodiment of the invention, as depicted in FIG. 3, comprises obtaining a nucleic acid sample from a subject; analyzing the nucleic acid sample, wherein analyzing comprises detecting the presence or absence of a target gene amplification product; and characterizing the subject as a silent carrier of SMA if the target gene amplification product is present.

In some embodiments, the method may comprise obtaining a nucleic acid sample from a subject. Nucleic acid may be obtained for conducting methods described herein without processing of the sample(s) containing the nucleic acid, in certain embodiments. In some embodiments, nucleic acid is obtained for conducting methods described herein after processing of the sample(s) containing the nucleic acid. For example, a nucleic acid can be extracted, isolated, purified, partially purified or amplified from the sample(s). The term “isolated” as used herein refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” as used herein can refer to a nucleic acid removed from a subject (e.g., a human subject). An isolated nucleic acid can be obtained with fewer non-nucleic acid components (e.g., protein, lipid) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” as used herein can refer to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components. The term “purified” as used herein can refer to a nucleic acid obtained that contains fewer nucleic acid species than in the sample source from which the nucleic acid is derived. A composition comprising purified nucleic acid may be about 90%, 91%, 92%, 93%, 94%, 95%, 96%, 97%, 98%, 99% or greater than 99% free of other nucleic acid species.

The method may further comprise analyzing the nucleic acid sample, wherein analyzing the nucleic acid sample comprises detecting the presence or absence of a target gene amplification product. The term “amplification product” as used herein refers to the product of subjecting a target nucleic acid in a sample to a process that linearly or exponentially generates amplicon nucleic acids having the same or substantially the same nucleotide sequence as the target nucleic acid, or segment thereof. The term “amplification product” as used herein can refer to the product of subjecting a target nucleic acid (e.g., in a sample comprising other nucleic acids) to a process that selectively and linearly or exponentially generates amplicon nucleic acids having the same or substantially the same nucleotide sequence as the target nucleic acid, or segment thereof. The term “amplification product” as used herein can refer to the product of subjecting a population of nucleic acids to a process that non-selectively and linearly or exponentially generates amplicon nucleic acids having the same or substantially the same nucleotide sequence as nucleic acids, or portions thereof, that were present in the sample prior to amplification. The method may further comprise characterizing the subject as a silent carrier of SMA if the target gene amplification product is present.

In some embodiments, the term “amplification product” refers to the product of a method that comprises a polymerase chain reaction (PCR). In some embodiments, the method of analyzing the nucleic acid sample may comprise a real-time PCR assay.

In some embodiments, the method of analyzing the nucleic acid sample may comprise analyzing exon 7 of SMN1 or a portion thereof. In some embodiments, the method of analyzing the nucleic acid sample may comprise analyzing rs143838139 in the SMN1 gene amplification product to detect the presence of a T>G mutation present in silent carriers. In some embodiments, primers comprising SEQ ID NO: 1 and SEQ ID NO: 2 are used to analyze exon 7 of SMN1 or a portion thereof.

| SEQ ID NO | Sequence |
|---|---|
| SEQ ID NO: 1 | CTTCCTTTATTTTCCTTACAGGGTTT [symbol] |
| SEQ ID NO: 2 | TTACATTAACCTTTCAACTTT [symbol] TAACATCTG |

In some embodiments, the method may comprise analyzing exon 8 of SMN1 or a portion thereof to identify a subject as a silent carrier of SMA. In some embodiments, the method of analyzing the nucleic acid sample may comprise analyzing the insertion/deletion variant rs200800214 (delAT MAF: 8.6% in gnomAD, 9.6% in 1000G) located in SMN1 exon 8.

In some embodiments of the method, a mutant SMN1 gene amplification product is detected with a labeled nucleic acid probe specific for the mutant SMN1 gene amplification product. In some embodiments, the labeled nucleic acid probe specific for the mutant SMN1 gene amplification product comprises SEQ ID NO: 3. In some embodiments of the method, a wild-type SMN1 gene amplification product is detected with a labeled nucleic acid probe specific for the wild-type SMN1 gene amplification product. In some embodiments, the labeled nucleic acid probe specific for the wild-type SMN1 gene amplification product comprises SEQ ID NO: 4.

| SEQ ID NO | Sequence |
|---|---|
| SEQ ID NO: 3 | 5′-ATGTTTTTGAACAGTTAAA-3′ |
| SEQ ID NO: 4 | 5′-ATGTTTTTGAACA [symbol] TTAAA-3′ |

In some embodiments, of the method, the probe may be labeled with a detectable moiety. In some embodiments, the detectable moiety is a fluorophore. In some embodiments, the fluorophore may be 6-carboxyfluorescein. In some embodiments, the fluorophore may be VIC. In some embodiments, each probe may be labeled with different fluorphores. Or, other detectable moieties such as those disclosed herein may be used.

In some embodiments of the method, presence of the polymorphism may be detected by the cycle number at which the fluorescent signal of the probe crosses a minimum threshold (Ct). In some embodiments, of the method, Ct is determined in the fluorescent dye channel after the data is collected, and the presence or absence of the allele is determined by whether the curve Ct is lower than a predetermined threshold.

In some embodiments, the subject is a human.

In some embodiments the sample is a biosample. In some embodiments, the sample is a blood sample or a portion of a blood sample such as serum or plasma.

### Compositions

Other embodiments of the disclosure include compositions for identifying a subject as a silent carrier of spinal muscular atrophy (SMA). The compositions may be embodied in a variety of ways.

In certain embodiments, disclosed is a composition for identifying a subject as a silent carrier of spinal muscular atrophy (SMA) by identifying the alleles present in the SMN1 gene copies in the subject. In an embodiment, the composition comprises components for analyzing rs143838139 in the SMN1 gene to detect the presence of a T>G mutation.

In certain embodiments, the compositions comprise a primer comprising the nucleic acid sequences of SEQ ID NO: 1 and/or SEQ ID NO: 2, or a nucleic acid sequence at least 99%, or 98%, or 97%, or 96%, or 95%, or 90%, or 85%, or 80% identical thereto. Additionally, and/or alternatively, the compositions comprise a probe comprising the nucleic acid sequences of SEQ ID NO: 3 and/or SEQ ID NO: 4, or a nucleic acid sequence at least 99%, or 98%, or 97%, or 96%, or 95%, or 90%, or 85%, or 80% identical thereto. In certain embodiments, the primer and/or probe may be labeled with a detectable moiety. In some embodiments, the detectable moiety is a fluorophore. In some embodiments, the fluorophore may be 6-carboxyfluorescein. In some embodiments, the fluorophore may be VIC. In some embodiments, each probe may be labeled with different fluorphores. Or, other detectable moieties such as those disclosed herein may be used.

Any of a wide variety of detectable agents can be used in the practice of the disclosure. Suitable detectable agents include, but are not limited to: various ligands, radionucleotides; fluorescent dyes; chemiluminescent agents (such as, for example, acridinum esters, stabilized dioxetanes, and the like); bioluminescent agents; spectrally resolvable inorganic fluorescent semiconductors nanocrystals (i.e., quantum dots); microparticles; metal nanoparticles (e.g., gold, silver, copper, platinum, etc.); nanoclusters; paramagnetic metal ions; enzymes; colorimetric labels (such as, for example, dyes, colloidal gold, and the like); biotin; dioxigenin; haptens; and proteins for which antisera or monoclonal antibodies are available.

Below are described some non-limiting examples of some detectable moieties that may be used.

### Fluorescent Dyes

In certain embodiments, a detectable moiety is a fluorescent dye. Numerous known fluorescent dyes of a wide variety of chemical structures and physical characteristics are suitable for use in the practice of the disclosure. A fluorescent detectable moiety can be stimulated by a laser with the emitted light captured by a detector. The detector can be a charge-coupled device (CCD) or a confocal microscope, which records its intensity.

Suitable fluorescent dyes include, but are not limited to, fluorescein and fluorescein dyes (e.g., fluorescein isothiocyanine or FITC, naphthofluorescein, 4′,5′-dichloro-2′,7′-dimethoxyfluorescein, 6-carboxyfluorescein or FAM, etc.), hexachloro-fluorescein (HEX), carbocyanine, merocyanine, styryl dyes, oxonol dyes, phycoerythrin, erythrosin, eosin, rhodamine dyes (e.g., carboxytetramethylrhodamine or TAMRA, carboxyrhodamine 6G, carboxy-X-rhodamine (ROX), lissamine rhodamine B, rhodamine 6G, rhodamine Green, rhodamine Red, tetramethylrhodamine (TMR), etc.), coumarin and coumarin dyes (e.g., methoxycoumarin, dialkylaminocoumarin, hydroxycoumarin, aminomethylcoumarin (AMCA), etc.), Q-DOTS, Oregon Green Dyes (e.g., Oregon Green 488, Oregon Green 500, Oregon Green 514., etc.), Texas Red, Texas Red-X, SPECTRUM RED, SPECTRUM GREEN, cyanine dyes (e.g., CY-3, CY-5, CY-3.5, CY5.5, etc.), ALEXA FLUOR dyes (e.g., ALEXA FLUOR 350, ALEXA FLUOR 488, ALEXA FLUOR 532, ALEXA FLUOR 546, ALEXA FLUOR 568, ALEXA FLUOR 594, ALEXA FLUOR 633, ALEXA FLUOR 660, ALEXA FLUOR 680, etc.), BODIPY dyes (e.g., BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665, etc.), IRDyes (e.g., IRD40, IRD 700, IRD 800, etc.), and the like. For more examples of suitable fluorescent dyes and methods for coupling fluorescent dyes to other chemical entities such as proteins and peptides, see, for example, “The Handbook of Fluorescent Probes and Research Products”, 9th Ed., Molecular Probes, Inc., Eugene, Oreg. Favorable properties of fluorescent labeling agents include high molar absorption coefficient, high fluorescence quantum yield, and photostability. In some embodiments, labeling fluorophores exhibit absorption and emission wavelengths in the visible (i.e., between 400 and 750 nm) rather than in the ultraviolet range of the spectrum (i.e., lower than 400 nm).

A detectable moiety may include more than one chemical entity such as in fluorescent resonance energy transfer (FRET). Resonance transfer results an overall enhancement of the emission intensity. For instance, see Ju et. al. (1995) Proc. Nat'l Acad. Sci. (USA) 92:4347, the entire contents of which are herein incorporated by reference. To achieve resonance energy transfer, the first fluorescent molecule (the “donor” fluor) absorbs light and transfers it through the resonance of excited electrons to the second fluorescent molecule (the “acceptor” fluor). In one approach, both the donor and acceptor dyes can be linked together and attached to the oligo primer. Methods to link donor and acceptor dyes to a nucleic acid have been described, for example, in U.S. Pat. No. 5,945,526 to Lee et al., the entire contents of which are herein incorporated by reference. Donor/acceptor pairs of dyes that can be used include, for example, fluorescein/tetramethylrohdamine, IAEDANS/fluroescein, EDANS/DABCYL, fluorescein/fluorescein, BODIPY FL/BODIPY FL, and Fluorescein/QSY 7 dye. See, e.g., U.S. Pat. No. 5,945,526 to Lee et al. Many of these dyes also are commercially available, for instance, from Molecular Probes Inc. (Eugene, Oreg.). Suitable donor fluorophores include 6-carboxyfluorescein (FAM), tetrachloro-6-carboxyfluorescein (TET), 2′-chloro-7′-phenyl-1,4-dichloro-6-carboxyfluorescein (VIC), and the like.

### Enzymes

In certain embodiments, a detectable moiety is an enzyme. Examples of suitable enzymes include, but are not limited to, those used in an ELISA, e.g., horseradish peroxidase, beta-galactosidase, luciferase, alkaline phosphatase, etc. Other examples include beta-glucuronidase, beta-D-glucosidase, urease, glucose oxidase, etc. An enzyme may be conjugated to a molecule using a linker group such as a carbodiimide, a diisocyanate, a glutaraldehyde, and the like.

### Radioactive Isotopes

In certain embodiments, a detectable moiety is a radioactive isotope. For example, a molecule may be isotopically-labeled (i.e., may contain one or more atoms that have been replaced by an atom having an atomic mass or mass number different from the atomic mass or mass number usually found in nature) or an isotope may be attached to the molecule. Non-limiting examples of isotopes that can be incorporated into molecules include isotopes of hydrogen, carbon, fluorine, phosphorous, copper, gallium, yttrium, technetium, indium, iodine, rhenium, thallium, bismuth, astatine, samarium, and lutetium (i.e., 3H, 13C, 14C, 18F, 19F, 32P, 35S, 64Cu, 67Cu, 67Ga, 90Y, 99mTc, 111In, 125I, 123I, 129I, 131I, 135I, 186Re, 187Re, 201T1, 212Bi, 213Bi, 21lAt, 153Sm, 177Lu).

### Dendrimers

In some embodiments, signal amplification is achieved using labeled dendrimers as the detectable moiety (see, e.g., Physiol Genomics 3:93-99, 2000), the entire contents of which are herein incorporated by reference in their entirety. Fluorescently labeled dendrimers are available from Genisphere (Montvale, N.J.). These may be chemically conjugated to the oligonucleotide primers by methods known in the art.

### Systems and Kits

Other embodiments of the disclosure include systems and kits for identifying a subject as a silent carrier of spinal muscular atrophy (SMA). The systems may be embodied in a variety of ways.

In certain embodiments, the system for identifying a subject as a silent carrier of SMA comprises at least one station or a component (e.g., a composition) for performing at least one of the following steps: (a) obtaining a nucleic acid sample from a subject; (b) analyzing the nucleic acid sample, wherein analyzing the nucleic acid sample comprises detecting the presence or absence of a target gene amplification product; and (c) characterizing the subject as a silent carrier of SMA if the target gene amplification product is present.

In certain embodiments, the kit for identifying a subject as a silent carrier of SMA comprises at least one a component (e.g., a composition) for performing at least one of the following steps: (a) obtaining a nucleic acid sample from a subject; (b) analyzing the nucleic acid sample, wherein analyzing the nucleic acid sample comprises detecting the presence or absence of a target gene amplification product; and (c) characterizing the subject as a silent carrier of SMA if the target gene amplification product is present. In certain embodiments, the composition used in the kit may comprise a primer comprising the nucleic acid sequences of SEQ ID NO: 1 and/or SEQ ID NO: 2, or a nucleic acid sequence at least 99%, or 98%, or 97%, or 96%, or 95%, or 90%, or 85%, or 80% identical thereto. Additionally, and/or alternatively, the compositions comprise a probe comprising the nucleic acid sequences of SEQ ID NO: 3 and/or SEQ ID NO: 4, or a nucleic acid sequence at least 99%, or 98%, or 97%, or 96%, or 95%, or 90%, or 85%, or 80% identical thereto. In certain embodiments, the primer and/or probe may be labeled with a detectable moiety. In some embodiments, the detectable moiety is a fluorophore. In some embodiments, the fluorophore may be 6-carboxyfluorescein. In some embodiments, the fluorophore may be VIC. In some embodiments, each probe may be labeled with different fluorphores. Or, other detectable moieties such as those disclosed herein may be used. In certain embodiments, the kit includes instructions for use.

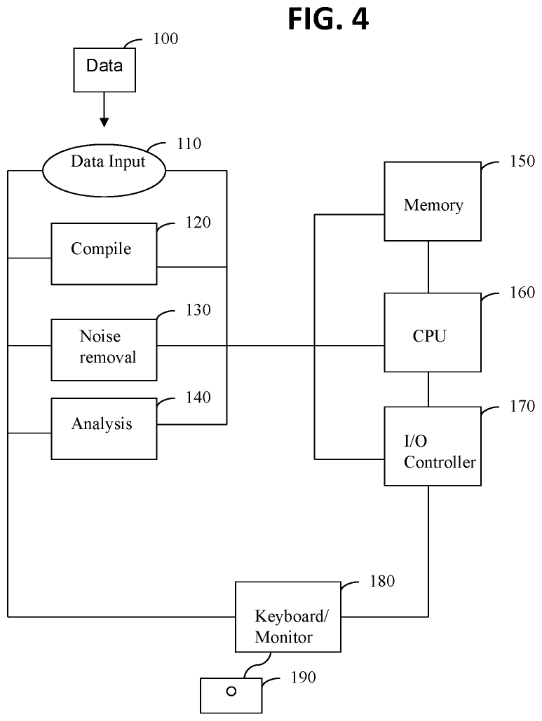

In some embodiments, the present disclosure provides systems for identifying a subject as a silent carrier of spinal muscular atrophy (SMA). Thus, in an embodiment, the present invention comprises a computer-readable medium on which is encoded programming code for the methods described herein. Also in an embodiment, present invention may comprise a system comprising a processor in communication with a computer-readable medium, the processor configured to perform the methods described herein. Suitable processors and computer-readable media for various embodiments of the present invention are described in greater detail below and are illustrated in FIG. 4.

Thus, in certain embodiments, the disclosure comprises a system for identifying a subject as a silent carrier of spinal muscular atrophy (SMA) comprising: a computer readable medium; and a processor in communication with the computer readable medium, the processor configured to identify a subject as a silent carrier of spinal muscular atrophy (SMA).

In other embodiments, the invention comprises a computer readable medium on which is encoded program code for predicting a subject as a silent carrier of spinal muscular atrophy (SMA), the program code comprising code for applying a model to estimate the effects of individual mutations in the at least one gene.

Some embodiments of the systems and computer readable media of the invention may be applied to various genes. In certain embodiments, the at least one gene comprises the SMN1 gene.

Embodiments in accordance with aspects of the present subject matter can be implemented in digital electronic circuitry, in computer hardware, firmware, software, or in combinations of the preceding. In one embodiment, a computer may comprise a processor or processors. The processor may comprise, or have access to, a computer-readable medium, such as a random access memory coupled to the processor. The processor may execute computer-executable program instructions stored in memory, such as executing one or more computer programs including a sampling routine and suitable programming to produce output to generate the analysis described in detail herein.

Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may comprise, or may be in communication with, media, for example tangible computer-readable media that may store instructions that when executed by the processor, can cause the processor to perform the steps described herein as carried out, or assisted, by a processor. Embodiments of computer-readable media may comprise, but are not limited to, all electronic, optical, magnetic, or other storage devices capable of providing a processor, such as the processor in a web server, with computer-readable instructions. Other examples of media comprise, but are not limited to, a floppy disk, CD-ROM, magnetic disk, memory chip, ROM, RAM, ASIC, configured processor, all optical media, all magnetic tape or other magnetic media, or any other medium from which a computer processor can read. Also, various other devices may include computer-readable media, such as a router, private or public network, or other transmission device. The processor, and the processing may be in one or more structures, and may be dispersed through one or more structures. The processor may comprise code for carrying out one or more of the methods (or parts of methods) described herein.

The system may comprise a data compiling system as well as a means for the user to interact with the system as the analysis proceeds. Thus, in an embodiment, the present invention may comprise a system for collecting and/or compiling data from a plurality of assay measurements and/or sequencing data and transmitting the data to a computer, and a system for transmitting the results of the analysis to a user. The systems may be designed for high-throughput analysis of DNA and/or amino acid sequencing data. Thus, in an embodiment, the plurality of measured signals comprise a plurality of known DNA sequences isolated from at least one cell type.

FIG. 4 shows an embodiment of the flow of information in a system comprising the software of the present invention. As discussed above, a computer processor or CPU may include, for example, digital logic processors capable of processing input, executing algorithms, and generating output as necessary in response to the inputs received from the touch-sensitive input device. As detailed herein, such processors may include a microprocessor, such as an ASIC, and state machines, and/or other components. Such processors include, or may be in communication with, media, for example computer-readable media, which stores instructions that, when executed by the processor, cause the processor to perform the steps described herein.

Thus, in an embodiment, the starting point may comprise data (100). Once the data has been collected (110), it may be compiled (120) and/or transformed if necessary, using the appropriate statistical analysis and/or standard spreadsheet software such as Microsoft Excel, FoxPro, Lotus, or the like prior to or as part of the analysis. In an embodiment, the data are entered into the system for each experiment. Alternatively, data from previous runs are stored in the computer memory (150) and used as required.

At each point in the analysis, the user may input instructions via a keyboard (180), floppy disk, remote access (e.g., via the internet) (190), or other access means. The user may enter instructions including options for the run, how reports should be printed out, and the like. Also, at each step in the analysis, the data may be stored in the computer using a storage device common in the art such as disks, drives or memory (150). As is understood in the art, the processor (160) and I/O controller (170) are required for multiple aspects of computer function. Also, in an embodiment, there may be more than one processor.

The data may also be processed to remove noise (130). In some cases, the user, via the keyboard (180), floppy disk, or remote access (190), may want to input variables or constraints for the analysis, as for example, the threshold for determining noise. The results of the analysis may then be compiled and provided in a form for review by a user (140).

### EXAMPLES

### Example 1—Method for Detecting an SMA Silent Carrier

The SMN1 silent carrier assay uses TaqMan real-time PCR technology to specifically target SMN1 and the NM_000344:c.*3+80T>G SNP. Briefly, two TaqMan probes labeled with either a FAM or VIC fluorophore are used to detect the presence of the polymorphic G variant or the wild-type T base, respectively. The upstream qPCR primer provides specificity to SMN1 at the most 3′ base, which is complementary to the SMN1-specific C base at chr5: 70247773 (hg19). Data collection and analysis are performed using the QuantStudio™ Real Time PCR software (v1.2, ThermoScientific) paired with the QS7 thermal cycling instrument (ThermoScientific). Presence of the polymorphism is detected by the cycle number at which its fluorescent signal crosses a minimum threshold (Ct). When no signal is detected for the polymorphism, the fluorescent signal from the WT probe is used as a sample-specific assay control.

In the SMN1 Silent Carrier assay, for the SMN1 target sequence the upstream PCR primer is designed to be specific for the SMN1 gene by targeting the C base at chr5:70247773 (GRCh37/hg19 build), which is used to discriminate SMN1 from the pseudogene, SMN2. The reverse PCR primer is also specific for the SMN1 gene. The SMN1 amplicon size is 139 bp. The primer and probe sequences are listed in Table 1.

TABLE 1
Primers and Probes used in the SMN1 Silent Carrier Assay

| Primer/ | | |
|---|---|---|
| probe name | Sequence | Genomic location (HG19) |
| MUT Probe | 5′FAM-ATGTTTTTGAACA [symbol] TTAAA- | chr5: 70247888-70247906 |
| | 3′MGB-NFQ | |
| WT Probe | 5′VIC-ATGTTTTTGAACA [symbol] TTAAA- | chr5: 70247888-70247906 |
| | 3′MGB-NFQ | |
| Forw | CTTCCTTTATTTTCCTTACAGGGTT | chr5: 70247747-70247773 |
| | T [symbol] | |
| Rev | TTACATTAACCTTTCAACTTT [symbol] AAC | chr5: 70247912-70247942 |
| | ATCTG | |

[symbol]

-base targeted by MUT/WT probe [symbol]
-base specific to SMN1 gene

Once the TaqMan data are collected, the Cₜ is determined for both fluorescent dye channels, and the data quality is evaluated. Presence or absence of the allele is determined by whether the curve Cₜ is lower than a predetermined threshold, and the quality of the curve is measured by the slope of the linear curve in the exponential amplification phase, which is determined by the equation:

$$slope = {log}_{10} \frac{( Δ {C}_{t + 3} - {ΔC}_{t + 1} )}{2} ,$$

where Cₜ₊₁ is the first whole cycle number after the Cₜ.

### Example 2—Examples of Certain Embodiments

Listed hereafter are non-limiting examples of certain embodiments of the technology.

A1. A method for identifying a subject as a silent carrier of spinal muscular atrophy (SMA) comprising:
(a) obtaining a nucleic acid sample from a subject;
(b) analyzing the nucleic acid sample, wherein analyzing the nucleic acid sample comprises detecting the presence or absence of a target gene amplification product; and
(c) characterizing the subject as a silent carrier of SMA if the target gene amplification product is present.

A2. The method of embodiment A1, wherein analyzing the nucleic acid sample comprises a real-time PCR assay.

A3. The method of any of embodiments A1 to A2, wherein analyzing the nucleic acid sample comprises analyzing exon 7 of SMN1 or a portion thereof.

A4. The method of any of embodiments A1 to A3, wherein primers comprising SEQ ID NO: 1 and SEQ ID NO: 2 are used to analyze exon 7 of SMN1 or a portion thereof.

A5. The method of any of embodiments A1 to A4, wherein analyzing the nucleic acid sample comprises analyzing rs143838139 in the SMN1 gene amplification product to detect the presence of a T>G mutation.

A6. The method of any of embodiments A1 to A5, wherein analyzing the nucleic acid sample comprises targeted analysis of rs143838139 in the SMN1 gene amplification product utilizing specific Taqman probes.

A7. The method of embodiment A6, wherein a wild type SMN1 gene amplification product is detected with a labeled nucleic acid probe specific for the wild type SMN1 gene amplification product.

A8. The method of embodiment A7, wherein the probe comprises SEQ ID NO: 3.

A9. The method of embodiment A6, wherein a mutant SMN1 gene amplification product is detected with a labeled nucleic acid probe specific for the mutant SMN1 gene amplification product.

A10. The method of embodiment A9, wherein the probe comprises SEQ ID NO: 4.

A11. The method of any of embodiments A6 to A10, wherein the two probes are labeled with different fluorophores.

A12. The method of any of embodiments A6 to A11, wherein Ct is determined for each fluorescent dye channel after the data is collected.

A13. The method of embodiment A12, wherein the presence or absence of the allele is determined by whether the curve Ct is lower than a predetermined threshold.

A14. The method of any of embodiments A1 to A13, wherein the subject is a human.

A15. The method of any of embodiments A1 to A14, wherein the sample is a blood sample.

B1. A system for identifying a subject as a silent carrier of spinal muscular atrophy (SMA) using the methods of any of the preceding embodiments.

B2. A system for identifying a subject as a silent carrier of SMA comprising at least one station or component for performing at least one of the following steps:
(a) obtaining a nucleic acid sample from a subject;
(b) analyzing the nucleic acid sample, wherein analyzing the nucleic acid sample comprises detecting the presence or absence of a target gene amplification product; and
(c) characterizing the subject as a silent carrier of SMA if the target gene amplification product is present.

C1. A kit for identifying a subject as a silent carrier of spinal muscular atrophy using the methods of any of the preceding embodiments.

C2. The kit of C1, comprising components for performing at least one of the following steps:
(a) obtaining a nucleic acid from a subject;
(b) analyzing the nucleic acid sample, wherein analyzing the nucleic acid sample comprising detecting the presence or absence of a target gene amplification product; and
(c) characterizing the subject as a silent carrier of SMA if the target gene amplification product is present.

C3. The kit of C1, further comprising a primer comprising the nucleic acid sequence of SEQ ID NO:1 or a nucleic acid sequence at least 99%, or 98%, or 97%, or 96%, or 95%, or 90%, or 85%, or 80% identical thereto.

C4. The kit of C1, further comprising a primer comprising the nucleic acid sequence of SEQ ID NO: 2 or a nucleic acid sequence at least 99%, or 98%, or 97%, or 96%, or 95%, or 90%, or 85%, or 80% identical thereto.

C5. The kit of C1, further comprising a probe comprising the nucleic acid sequence of SEQ ID NO: 3 or a nucleic acid sequence at least 99%, or 98%, or 97%, or 96%, or 95%, or 90%, or 85%, or 80% identical thereto.

C6. The kit of C1, further comprising a probe comprising the nucleic acid sequence of SEQ ID NO: 4 or a nucleic acid sequence at least 99%, or 98%, or 97%, or 96%, or 95%, or 90%, or 85%, or 80% identical thereto.

C7. The kit of any of embodiments C2 to C6, wherein the primer and/or probe may be labeled with a detectable moiety.

C8. The kit of embodiment C7, wherein the detectable moiety is a fluorophore, optionally at least one of 6-carboxyfluorescein or VIC.

D1. A composition for identifying a subject as a silent carrier of spinal muscular atrophy (SMA) by identifying the alleles present in the SMN1 gene copies in the subject.

D2. The composition of embodiment D1, comprising components for analyzing rs143838139 in the SMN1 gene to detect the presence of a T>G mutation.

D3. The composition of the embodiment of D1 and/or D2, comprising a primer comprising the nucleic acid sequences of SEQ ID NO: 1 and/or SEQ ID NO: 2, or a nucleic acid sequence at least 99%, or 98%, or 97%, or 96%, or 95%, or 90%, or 85%, or 80% identical thereto.

D4. The composition of the embodiment of D1 and/or D2, comprising a probe comprising the nucleic acid sequences of SEQ ID NO: 3 and/or SEQ ID NO: 4, or a nucleic acid sequence at least 99%, or 98%, or 97%, or 96%, or 95%, or 90%, or 85%, or 80% identical thereto.

D5. The composition of the embodiment of D3 and/or D4, wherein at least one of the primer and/or probe is labeled with a detectable moiety.

D6. The composition of the embodiment of D5, wherein the detectable moiety is a fluorophore, optionally at least one of 6-carboxyfluorescein or VIC.

That which is claimed is:

1. A method for identifying a subject as a silent carrier of spinal muscular atrophy (SMA) comprising:
(a) obtaining a nucleic acid sample from a subject;
(b) analyzing the nucleic acid sample, wherein analyzing the nucleic acid sample comprises detecting the presence or absence of a target gene amplification product; and
(c) characterizing the subject as a silent carrier of SMA if the target gene amplification product is present.

2. The method of claim 1, wherein analyzing the nucleic acid sample comprises a real-time PCR assay.

3. The method of claim 1, wherein analyzing the nucleic acid sample comprises analyzing exon 7 of SMN1 or a portion thereof.

4. The method of claim 3, wherein primers comprising SEQ ID NO: 1 and SEQ ID NO: 2 or a nucleic acid sequence at least 99%, or 98%, or 97%, or 96%, or 95%, or 90%, or 85%, or 80% identical thereto are used to analyze exon 7 of SMN1 or a portion thereof.

5. The method of claim 1, wherein analyzing the nucleic acid sample comprises analyzing rs143838139 in the SMN1 gene amplification product to detect the presence of a T>G mutation.

6. The method of claim 1, wherein analyzing the nucleic acid sample comprises targeted analysis of rs143838139 in the SMN1 gene amplification product utilizing specific Taqman probes.

7. The method of claim 6, wherein a wild type SMN1 gene amplification product is detected with a labeled nucleic acid probe specific for the wild type SMN1 gene amplification product.

8. The method of claim 7, wherein the probe comprises SEQ ID NO: 3 or a nucleic acid sequence at least 99%, or 98%, or 97%, or 96%, or 95%, or 90%, or 85%, or 80% identical thereto.

9. The method of claim 6, wherein a mutant SMN1 gene amplification product is detected with a labeled nucleic acid probe specific for the mutant SMN1 gene amplification product.

10. The method of claim 9, wherein the probe comprises SEQ ID NO: 4 or a nucleic acid sequence at least 99%, or 98%, or 97%, or 96%, or 95%, or 90%, or 85%, or 80% identical thereto.

11. The method of claim 6, wherein the two probes are labeled with different fluorophores.

12. The method of claim 11, wherein Ct is determined for each fluorescent dye channel after the data is collected.

13. The method of claim 12, wherein the presence or absence of the allele is determined by whether the curve Ct is lower than a predetermined threshold.

14. The method of claim 1, wherein the subject is a human.

15. The method of claim 1, wherein the sample is a blood sample.

16. A system for identifying a subject as a silent carrier of spinal muscular atrophy (SMA), the system comprising at least one station or component for performing at least one of the following steps:
(a) obtaining a nucleic acid sample from a subject;
(b) analyzing the nucleic acid sample, wherein analyzing the nucleic acid sample comprises detecting the presence or absence of a target gene amplification product; and
(c) characterizing the subject as a silent carrier of SMA if the target gene amplification product is present.

17. A kit for identifying a subject as a silent carrier of spinal muscular atrophy (SMA), wherein the kit comprises components for performing at least one of the following steps:
(a) obtaining a nucleic acid from a subject;
(b) analyzing the nucleic acid sample, wherein analyzing the nucleic acid sample comprising detecting the presence or absence of a target gene amplification product; and
(c) characterizing the subject as a silent carrier of SMA if the target gene amplification product is present.

18. A composition for identifying a subject as a silent carrier of spinal muscular atrophy (SMA) by identifying the alleles present in the SMN1 gene copies in the subject, wherein the composition comprises components for analyzing rs143838139 in the SMN1 gene to detect the presence of a T>G mutation.

19. The composition of claim 18, comprising a primer comprising the nucleic acid sequences of SEQ ID NO: 1 and/or SEQ ID NO: 2, or a nucleic acid sequence at least 99%, or 98%, or 97%, or 96%, or 95%, or 90%, or 85%, or 80% identical thereto.

20. The composition of claim 18, comprising a probe comprising the nucleic acid sequences of SEQ ID NO: 3 and/or SEQ ID NO: 4, or a nucleic acid sequence at least 99%, or 98%, or 97%, or 96%, or 95%, or 90%, or 85%, or 80% identical thereto.


SEQUENCE LISTING (4 sequences)

SEQ ID NO: 1 - DNA, 27 bp, Artificial Sequence
  source: /note=“Description of Artificial Sequence Synthetic primer”
cttcctttattttccttacagggtttc

SEQ ID NO: 2 - DNA, 31 bp, Artificial Sequence
  source: /note=“Description of Artificial Sequence Synthetic primer”
ttacattaacctttcaactttttaacatctg

SEQ ID NO: 3 - DNA, 19 bp, Artificial Sequence
  source: /note=“Description of Artificial Sequence Synthetic probe”
atgtttttgaacagttaaa

SEQ ID NO: 4 - DNA, 19 bp, Artificial Sequence
  source: /note=“Description of Artificial Sequence Synthetic probe”
atgtttttgaacatttaaa